(19) United States
(12) Patent Application Publication
(10) Pub. No.: US 2023/0335238 A1
(43) Pub. Date: Oct. 19, 2023

(54) METHOD FOR TRANSMITTING DATA FROM A MEDICAL APPARATUS, AND MEDICAL APPARATUS

(71) Applicant: WEINMANN EMERGENCY MEDICAL TECHNOLOGY GMBH + CO. KG, Hamburg (DE)

(72) Inventor: Nils DANCKWARDT, Hamburg (DE)

(21) Appl. No.: 18/023,881

(22) PCT Filed: May 25, 2021
(86) PCT No.: PCT/DE2021/100451

§ 371 Date: Feb. 28, 2023

(30) Foreign Application Priority Data
Sep. 1, 2020 (DE) 10 2020 122 781.7

(51) Int. Cl. G16H 10/60; A61B 5/00

(52) U.S. Cl. CPC G16H 10/60; A61B 5/0022; G16H 40/63

(57) ABSTRACT

A method for transmitting data from a medical apparatus and to a medical apparatus. The transmission of data from medical apparatuses is made possible without a dedicated network connection to a digital database, using graphical 2D codes and a device for transmitting data from a medical apparatus, the device being in the form of, for example, a commercially available smartphone or tablet having a corresponding application for reading the 2D code and having a connection to a network.

The invention relates to a method for transmitting data from a medical apparatus to a digital database.

In addition, the invention relates to a medical apparatus, from which data are transmittable to a digital database.

During the use of medical apparatuses, for example ventilators, patient-related and also apparatus-related data arise, the storage of which in a database and/or further evaluation of which is desirable.

A connection to a local or a mobile network is often not provided during the use of medical apparatuses due to a lack of availability or a lack of apparatus equipment, however, so that easy transmission of data into a digital database is not possible.

A network in the meaning of this application is a network for the digital exchange of data, for example a wired or wireless local network (LAN/WLAN) or a location-spanning network, such as the Internet.

It is desirable to also be able to integrate patient-related data from such medical apparatuses into local or global digital databases, which do not have a separate connection to a network.

With the aid of such digital databases for patient-related data, it is possible for medical technicians and/or technical support facilities to carry out a central and digital evaluation of relevant vital parameters of a patient and/or treatments carried out on the patient.

It is furthermore also desirable to be able to integrate apparatus-related data from such medical apparatuses in local or global digital databases, which do not have a separate connection to a network.

With the aid of such digital databases for apparatus-related data, it is possible for the operator and/or the producer of the medical apparatuses to manage and/or evaluate status and/or usage data of the respective apparatuses, so that both proactive maintenance of hardware and software of the medical apparatuses and also proactive improvement of the hardware or software by the producer are enabled via updates or new developments.

The improvement of medical apparatuses on the basis of a variety of evaluable data enables systematic medical progress, which ultimately benefits the patients, who are dependent on a medical treatment with the aid of corresponding medical apparatuses.

However, receiving additional assemblies, which enable a connection of the apparatus to a local or a mobile network, in the medical apparatus is not always possible, in particular also with regard to mobile medical apparatuses, for reasons of cost and/or space.

In addition, a direct network connection of medical apparatuses is not always desirable from safety aspects (cybersecurity).

One object of the invention is therefore to specify a medical apparatus which enables the transmission of patient-related and/or apparatus-related data even without a separate connection to a network.

This object is achieved according to the invention by a medical apparatus as claimed in claim 1.

In addition, it is an object of the invention to specify a method for transmitting data from a medical apparatus to a digital database, by which a data transmission is made possible even if the medical apparatus does not have a connection to a network.

This object is achieved according to the invention by a method for transmitting data from a medical apparatus as claimed in claim 5.

The features described hereinafter of a medical apparatus and a method for transmitting data from a medical apparatus are each part of the invention both individually and also in all implementable combinations.

The use of visually coded data for transmitting the data from a medical apparatus to a digital database forms the foundation of the invention.

In one preferred embodiment of the invention, coding of the data with the aid of a visual two-dimensional code (2D code), such as at least one barcode, particularly preferably at least one QR code (quick response code) is used as the visual coding of the data.

The use of 2D codes designed as barcodes or QR codes has the advantage of the readability of these codes using a large number of apparatuses and applications available on the market.

According to the invention, however, other visual 2D codes are usable, which are designed, for example, in grayscales or multicolored and/or use differently shaped code elements instead of classic square pixels and/or have other external contours (for example rectangular or round).

In embodiments of the invention, a QR code has, according to specification, at least 21×21 and at most 177×177 elements in each symbol provided by a square (pixel) and in particularly preferred embodiments of the invention has an edge zone comprising at least four elements without pixels of the visual coding containing items of information.

In further embodiments of the invention, QR codes according to other specifications are used, such as micro QR codes, secure QR codes (SQRC), iQR codes, or frame QR codes.

A medical apparatus in the meaning of this invention is designed, for example, as a ventilator, a defibrillator, and/or as a measuring apparatus for at least one vital parameter.

A medical apparatus in the meaning of this invention has, in addition to the components required for the medical functionality and possibly medical modules, at least one memory unit, a control unit, a coding unit, and a display, wherein these assemblies can be entirely or partially provided especially for the use as a data transmission interface or for joint use with the medically required functionalities of the medical apparatus.

The more symbols are displayable in a 2D code with the aid of the display, the more data can be transmitted using a single 2D code.

The display of the medical apparatus has, in one preferred embodiment of the invention, a minimum resolution of 11×11 pixels, so that at least one 2D code designed as a micro QR code without edge zone is displayable.

Furthermore, embodiments are preferred having a display resolution of at least 29×29 pixels, so that at least one regular QR code having 21×21 symbols and a 4-pixel-wide edge area is displayable.

Embodiments having a display resolution of at least 180×180 pixels are particularly preferred, so that at least one regular QR code having 177×177 symbols and an edge area at least three pixels wide is displayable.

In other embodiments of the invention, in addition to the 2D code, further items of information or symbols are also displayable at the same time for the user of the medical apparatus, so that the above-mentioned minimum resolutions apply for the display area remaining for the display of the 2D code.

In particular patient-related data and/or apparatus-related data are considered data to be transmitted according to the invention.

In one embodiment of the invention, patient-related data are visually displayable by the medical apparatus with the aid of a 2D code on a display of the apparatus.

Such patient-related data can be in particular vital parameters of the patient acquired with the aid of the medical apparatus, person-related data of the patient, which have been input into the medical apparatus, or type and/or duration of a treatment of the patient carried out with the aid of the medical apparatus.

Patient-related data can be transmitted with the aid of the display of the 2D code on the display of the medical apparatus, for example, into a digital patient file of a patient, which is managed locally, for example in a hospital or a medical office, or independently thereof.

In one embodiment of the invention, apparatus-related data are visually displayable by the medical apparatus with the aid of a 2D code on a display of the apparatus.

Such apparatus-related data can be in particular an apparatus ID (apparatus identification number), such as a serial number, as well as operating data, status data, and/or service data.

Operating data are to be understood here as those data which specify the type and/or number of the execution of specific functions of the medical apparatus (operating modes and counter readings), such as the number of shocks given using a defibrillator, the number of charge cycles of the accumulator and/or a capacitor (defibrillator), the total number of operating hours, and/or the number of measurement cycles carried out of specific vital parameters.

Status data are to be understood here as those data which indicate the operational readiness of the medical apparatus as a whole or individual modules of the medical apparatus. Defects of individual modules can thus be specified, for example.

Service data are to be understood here as those data which relate to the equipment of the medical apparatus with hardware and/or software or errors of the medical apparatus occurring in operation. The hardware equipment can be specified here by the specification as to whether specific modules are equipped and which hardware version of the respective module is installed. The software equipment can be specified by the specification of the software version of the base system and possibly the additional modules and if necessary additional software packages installed on the medical apparatus. The errors which have possibly occurred in operation of a medical apparatus can be specified by an error log, which specifies, for example, an error number to identify the error and its frequency.

In embodiments of the invention, multiple codes can be displayed in succession to transmit larger amounts of data.

A medical apparatus according to the invention has a memory unit, in which patient-related and/or apparatus-related data are stored during the operation of the medical apparatus.

The data stored in the memory unit are readable and transferable to a coding unit with the aid of a control unit.

In preferred embodiments of the invention, data are directly readable from at least one medical module with the aid of the control unit, without these data having to be stored in the memory unit.

The data can be coded at least visually to form a 2D code with the aid of the coding unit.

The 2D code that can be generated with the aid of the coding unit contains in one preferred embodiment of the invention at least one URL, which in addition to an Internet address also contains the data to be transmitted.

In particularly preferred embodiments of the invention, a part of the data can additionally be encrypted using a key with the aid of the coding unit, so that these data can only be decoded if the key used for encrypting the data is known. This additional coding step suggests itself in particular for the transmission of sensitive data, for example patient data.

With the aid of the control unit, the display is actuatable to display the 2D code after the generation of the 2D code.

As soon as the data to be transmitted are displayed as a 2D code on the display of the medical apparatus according to the invention, they are readable with the aid of a device according to the invention for transmitting data from a medical apparatus and are transmittable to a digital database.

A device for transmitting data from a medical apparatus has for this purpose at least one acquisition unit for acquiring the 2D code, a decoding unit for decoding the 2D code, and a transmission unit for transmitting the data decoded from the 2D code to a digital database.

The acquisition unit is designed as a camera in embodiments of the invention. The transmission unit is designed in advantageous embodiments of the invention to establish a connection to a network and to transmit the data via this network to a digital database.

The digital database is implemented in embodiments of the invention on a local server.

In other embodiments of the invention, the digital database is implemented on a Web server.

A device used for the invention for transmitting data from a medical apparatus is designed, for example, as a smart phone or a tablet, on which an application for decoding a 2D code is installed so it is executable and which is connected to a network for transmitting the data to a predetermined digital database.

In the case of an implementation of the digital database on a local server, the device for transmitting data has access to the local network, to which the local server is connected. In the case of the implementation of the digital database on a Web server, the device for transmitting data has a connection to the Internet.

A method according to the invention for transmitting data of a medical apparatus includes at least the following steps:
reading defined data from a memory unit of the medical apparatus and/or retrieving defined data from at least one medical module of the medical apparatus
coding the data with the aid of a coding unit to form a graphic 2D code
displaying the generated 2D code on a display of the medical apparatus
acquiring the 2D code using a device for transmitting data of a medical apparatus
decoding the data from the graphic 2D code
transmitting the data to a predetermined digital database via a network

In embodiments of the invention, the following functions can be implemented individually or in combination with one another as additional method steps:
compressing the data read from the memory unit of the medical apparatus
generating a URL (uniform resource locator), which contains a link to a website and the data to be transmitted (after the data are read from the memory unit and/or at least one medical module and before the data are coded to form a 2D code)
opening a web browser and retrieving the URL (after the decoding of the data from the 2D code, partial functional overlap with the transmission of the data to a predetermined digital database)
encrypting data with the aid of a key (after the data are read from the memory unit and/or at least one medical module and before the data are coded to form a 2D code and possibly before the generation of a URL), and decrypting the encrypted data (after the transmission of the data to the predetermined digital database via a network)

In one preferred embodiment of the invention, a URL is generated, which specifies the connection to the predetermined digital database via the Internet with the aid of the secure hypertext transfer protocol (HTTPS). This has the advantage that the possibly sensitive data are (additionally) transmitted encrypted via the Internet.

Such a URL is composed as follows in advantageous embodiments of the invention: https://SERVERPATH/INSTRUCTIONS/DATA

The block “SERVERPATH” specifies the address of the server at which the digital database is located, to which the data are to be transmitted.

The block “INSTRUCTIONS” is optional and contains optional commands (for example display of the QR code or other items of information, language, such as PHP).

The data contained in the block “DATA” begin in advantageous embodiments of the invention with an identifier, which uniquely identifies the apparatus from which the data are transmitted, followed by the other data to be transmitted.

In preferred embodiments of the invention, at least a part of the data contained in the block “DATA” is encrypted with the aid of the coding unit of the medical apparatus.

In one particularly preferred embodiment of the invention, a part of the data is readable with the aid of the device for transmitting data from a medical apparatus and another part of the data is encrypted so that it is not readable with the aid of the device for transmitting data from a medical apparatus, at least without knowing the key used for the encryption.

It is thus possible to make data which are to be displayed to a user with the aid of the device for transmitting data from a medical apparatus readable to this user and to make those data which are not to be displayed to the user with the aid of the device for transmitting data from a medical apparatus only readable with the aid of the digital database or a suitable application, which accesses the digital database.

In one advantageous embodiment of the invention, a versioned layout of the 2D code is used, so that it can always be established for the decoding according to which pattern the data contained in a 2D code are constructed. For example, depending on the versioning, the sequence and/or number of the various data to be transmitted and/or the number of the characters used in each case for a specific data are defined, so that from a mere concatenation of the respective values, these are readable from the data packet and are to be assigned to the respective parameters.

In one preferred embodiment of the invention, 2D codes having an error correction are used (at least error correction level L in the case of QR codes), so that an erroneous transmission of the 2D code is detectable and correctable if necessary.

In advantageous embodiments of the invention, a feedback channel is implemented between the digital database and the server, on which the digital database is located, and the device for transmitting data from a medical apparatus, via which items of information are transferred to a user of the device for transmitting data from a medical apparatus and can be output thereto with the aid of the device for transmitting data from a medical apparatus.

Such items of information are provided in embodiments of the invention as the result of the transmission process of the data to the digital database (for example, successful/unsuccessful) and/or apparatus-specific items of information.

Apparatus-specific items of information can relate here, for example, to the operational readiness of the medical apparatus or individual modules of the medical apparatus (e.g., defects, faulty or absent connections, required maintenance).

In preferred embodiments of the invention, the apparatus-specific items of information are displayed so they are readable for a user, for example by the output of texts with the aid of a display.

In particularly preferred embodiments of the invention, the operating status of the medical apparatus is additionally indicated by color on the displays of the device for transmitting data from a medical apparatus (e.g., “green”=ready for operation, “red”=not ready for operation, “yellow”=maintenance/update required).

In particularly preferred embodiments of the invention, measures possibly required for maintenance or an update and/or to reestablish the operational readiness of the medical apparatus are displayed to the user on the display of the device for transmitting data from a medical apparatus.

In preferred embodiments of the invention having an implemented feedback channel to transfer items of information from the server via the device for transmitting data from a medical apparatus to the user, the method includes the following method steps individually or in combination with one another:
defining the data to be transmitted to the device for transmitting data from a medical apparatus on the server
transmitting the data from the server to the device for transmitting data from a medical apparatus
displaying the data transmitted from the server with the aid of the device for transmitting data from a medical apparatus (for example by display of a website)
displaying a code with the aid of the device for transmitting data from a medical apparatus and manually inputting this code at the medical apparatus by way of the user
checking the user input at the medical apparatus with the aid of a checking algorithm
outputting an input confirmation to the user

In one embodiment of the invention having an implemented feedback channel from the server to the device for transmitting data, a regular check of the operational readiness of the medical apparatus is implemented in such a way that the medical apparatus queries or requests a transmission of data to the server at regular intervals, in that a corresponding query is displayed on the display of the medical apparatus.

Upon a confirmation of the query by a user, the 2D code is generated and displayed, which contains the corresponding data. The data are evaluated on the server with respect to the operational readiness of the medical apparatus after the transmission with the aid of a device for transmitting data to the server and feedback about the status of the medical apparatus is output to the user with the aid of the device for transmitting data and/or the medical apparatus.

The situations mentioned as examples hereinafter provide an occasion for displaying a 2D code and the transmission connected thereto of data of the medical apparatus:
completing the function check of the medical apparatus
completing a software update of the medical apparatus
registering the medical apparatus
service case (apparatus fault of the medical apparatus)
upload device settings of the medical apparatus to the server (goal: operator can compare/track apparatus settings)
display device ID of the medical apparatus upon option purchase (acquire additional optional software)
complete an action using the medical apparatus→transmit the essential action-relevant data (→possible interface for data management)
sanity checks: for example, does the medical apparatus have the correct settings? Is the correct time of day set?
retrieval by a user

Exemplary embodiments and aspects of the invention are illustrated by way of example in the figures described hereinafter. In the figures:

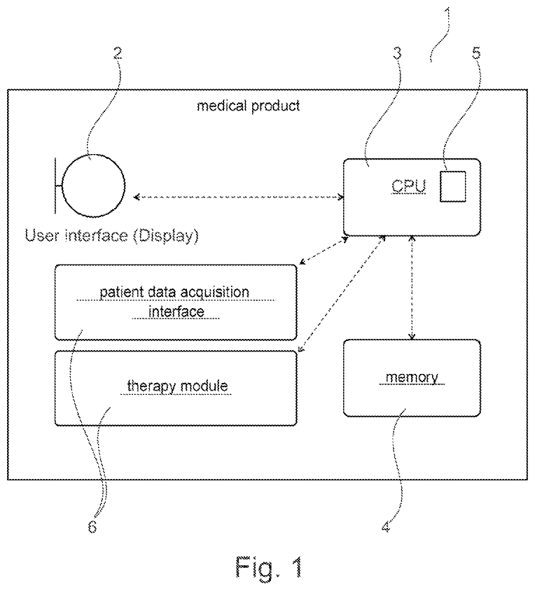

FIG. 1: shows a schematic block diagram of a medical apparatus according to the invention,

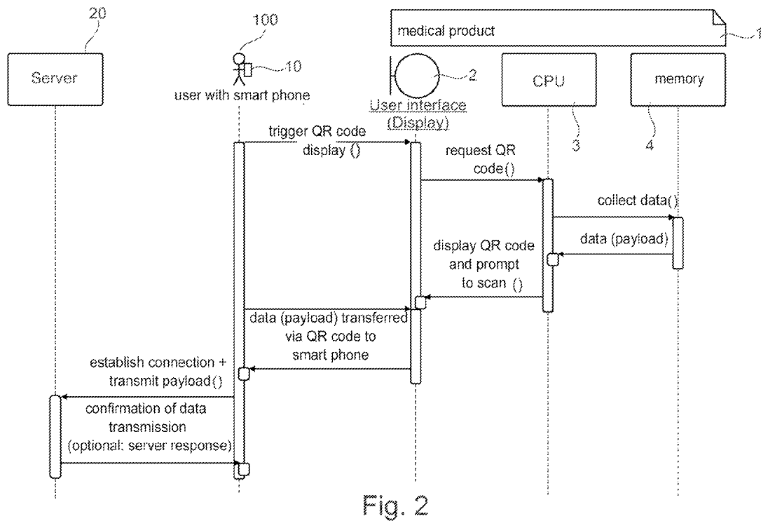

FIG. 2: shows a flow chart of a method according to the invention for transmitting data from a medical apparatus,

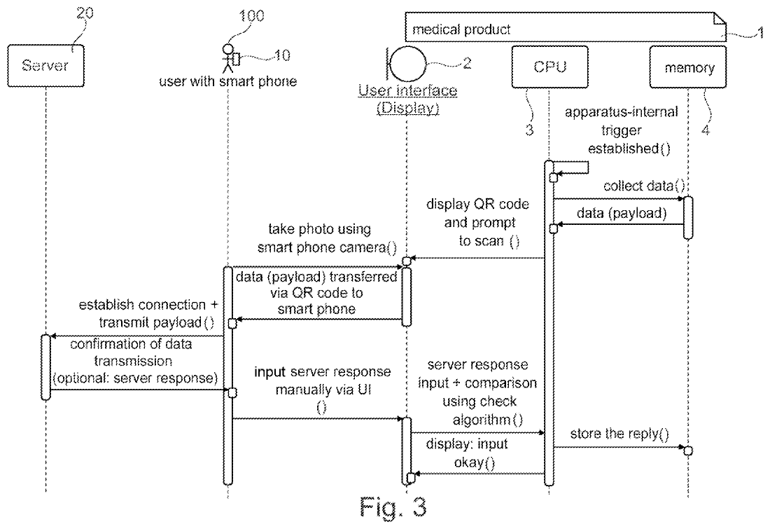

FIG. 3: shows a flow chart of a further aspect of an embodiment according to the invention of a method for transmitting data from a medical apparatus,

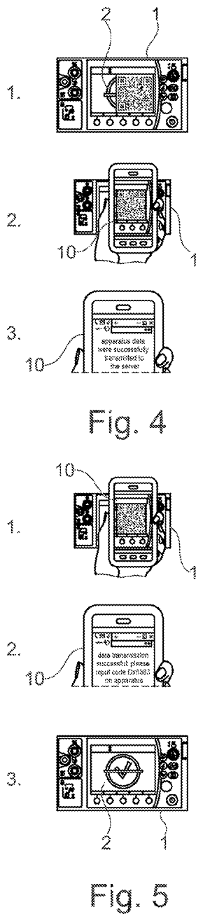

FIG. 4: shows a schematic sequence of an embodiment of a method according to the invention for transmitting data with differentiation of the method steps in the upload of the data visible to the user and running in the background, and

FIG. 5: shows a schematic sequence of a method according to FIG. 4 with reply.

FIG. 1 shows a schematic block diagram of at least a part of a medical apparatus (1) according to the invention. The medical apparatus (1) has a user interface designed as a display (2), a control unit (2) designed as a CPU, a memory unit (4) designed as a memory, and a coding unit (5) designed as a part of the control unit (3). In addition, the medical apparatus (1) has two medical modules (6), of which one module (6) is designed to acquire at least one vital parameter of a patient (patient data acquisition interface) and one module (6) is designed for therapeutic treatment of a patient (therapy module).

With the aid of the control unit (3), data acquired by the medical modules (6) and stored in the memory unit (4) are readable from the memory unit (4) and convertible with the aid of the coding unit (5) into a 2D code, which can be output with the aid of the display (2).

FIG. 2 shows a flow chart of a method according to the invention for transmitting data from a medical apparatus (1). In the exemplary embodiment shown, the medical apparatus (1) is triggered by a user (100) using a device for transmitting data from a medical apparatus (10), designed as a smart phone, to output a 2D code designed as a QR code on the display (2), for example, by a user input via a touch display or other input units (not shown) (such as buttons, keys, or rotary switches). The 2D code is thereupon requested and can retrieve various data depending on the user input.

The command to request a 2D code is transmitted to the control unit (3), which thereupon reads the required data out of the memory unit (4) and/or retrieves them directly from the medical modules (6). The data are accordingly transmitted to the control unit (3), where the data are coded with the aid of the coding unit (5) to generate a 2D code. In addition, an encryption of at least a part of the data preferably takes place. The 2D code contains a URL, which in turn contains the data to be transmitted.

The 2D code thus generated is displayed using the display (2) of the medical apparatus (1) and subsequently scanned from the display (2) by the user (100) with the aid of the device for transmitting data from a medical apparatus (10).

At least one application, which reads the 2D code and opens the URL, is executed here on the device for transmitting data from a medical apparatus (10). By opening the URL, which contains the address of the server (20) on which the digital database is placed, to which the data are to be transmitted, the data are transmitted by the device for transmitting data from a medical apparatus (10) to the digital database.

As an optional additional method step in the illustrated exemplary embodiment of the method according to the invention, a reply from the server (20) to the device for transmitting data from a medical apparatus (10) is provided, using which the data transmission is confirmed.

FIG. 3 shows a feedback channel, provided with additional functions, from the server (20) via the device for transmitting data from a medical apparatus (10) to the medical apparatus (1), wherein the server response is displayed to the user (100) on the device for transmitting data from a medical apparatus (10) and this user manually performs the corresponding inputs on the medical apparatus (1).

The input performed manually by the user (100) on the medical apparatus (1) is compared with the aid of the control unit (2) to a checking algorithm, the reply is stored in the memory unit (4) and a display, which confirms the input, is output on the display (2) of the medical apparatus (1) to the user (100).

FIG. 4 shows a schematic sequence of an embodiment of a method according to the invention for transmitting data with differentiation of the method steps visible to a user and those running in the background in the upload of the data.

In step 1, a medical apparatus (1) displays a 2D code designed as a QR code on a display. The medical apparatus (1) is shown in the center. The steps required, depending on the reason or trigger for the display of the QR code, previously run in the background. According to step 2, a user scans the display (2) of the medical apparatus (1) with the aid of a device for transmitting data from a medical apparatus (10) designed as a smart phone and a website is opened. Upon opening of the website, in the background the data to be transmitted are read from the QR code and transmitted to the website or the server (not shown). In step 3, the website is displayed on the smart phone, wherein it can be determined ad hoc with the aid of the server or software installed on the server which contents are displayed to the user.

FIG. 5 shows a schematic sequence of a method according to the invention as shown in FIG. 4, but with respect to the aspect of an implemented feedback channel from the server. Step 1 corresponds here to step 2 from FIG. 4. According to step 2, the website is displayed on the smart phone and the user is prompted to input a response to the medical apparatus (1). In the background, the server or software installed on the server defines the reply on the basis of the previously transmitted data. In step 3, the input of the server response is confirmed by the medical apparatus (1), in the exemplary embodiment shown by an optical output (check on the display). In the background, the medical apparatus (1) checks whether the reply matches a defined check pattern and executes corresponding functions if necessary.

1-11. (canceled)

12. A medical apparatus, comprising: at least one control unit; a memory unit; a display; at least one medical module configured to treat a patient or to acquire at least one vital parameter of the patient, wherein the control unit is configured to read predetermined patient-related and/or apparatus-related data from the memory unit and/or retrieve the data from at least one medical module; and a coding unit configured to code the data to form a 2D code, wherein the generated 2D code is output by the display.

13. The medical apparatus according to claim 12, wherein the display has a minimum resolution of x pixels.

14. The medical apparatus according to claim 12, wherein the 2D code is a QR code.

15. The medical apparatus according to claim 12, wherein the coding unit is additionally configured to encrypt at least a part of the.

16. A method for transmitting data from a medical apparatus to a digital database on a server, comprising the steps of:
reading defined data from a memory unit and/or retrieving defined data from at least one medical module of the medical apparatus;
coding the data with a coding unit to form a graphic 2D code;
displaying the generated 2D code on a display of the medical apparatus;
acquiring the 2D code using a device for transmitting data of a medical apparatus;
decoding the data from the graphic 2D code; and
transmitting the data to a predetermined digital database via a network.

17. The method according to claim 16, further comprising the step of compressing the data read from the memory unit of the medical apparatus and/or the data read from at least one medical module.

18. The method according to claim 16, further comprising the step of generating a URL that contains a link to a website and the data to be transmitted after the data are read from the memory unit and/or at least one medical module and before the data are coded to form a 2D code.

19. The method according to claim 18, further comprising the step of opening a web browser and retrieving the URL after the decoding of the data from the 2D code, partial functional overlap with the transmission of the data to a predetermined digital database.

20. The method according to claim 18, further comprising this steps of: encrypting the data with a key after the data are read from the memory unit and/or at least one medical module and before the data are coded to form a 2D code and optionally before the URL is generated: and decrypting the encrypted data after the data are transmitted to the predetermined digital database via the network.

21. The method according to claim 16, wherein the transmitting step includes implementing a feedback channel via which items of information with respect to a completed communication and/or selected apparatus-specific data are transmitted to the device for transmitting data from the medical apparatus, and outputting these items of information to a user of the device for transmitting data from the medical apparatus.

22. The method according to claim 16, including using a medical apparatus comprising: at least one control unit; the memory unit; the display; at least one medical module configured to treat a patient or to acquire at least one vital parameter of the patient, wherein the control unit is configured to read the predetermined patient-related and/or apparatus-related data from the memory unit and/or retrieve the data from the at least one medical module; and the coding unit configured to code the data to form the 2D code, wherein the generated 2D code is output by the display.
